(19) United States
(12) Patent Application Publication
(10) Pub. No.: US 2010/0069928 A1
(43) Pub. Date: Mar. 18, 2010

(54) ANCHOR, SYSTEM AND METHOD TO ATTACH A HUMAN TISSUE OR SUTURE TO A BONE

(76) Inventor: Alberto Bauer, Nueva Andalucia (Marbella) (IT)

Correspondence Address: K.F. ROSS P.C., 5683 RIVERDALE AVENUE, SUITE 203 BOX 900, BRONX, NY, 10471-0900, US

(21) Appl. No.: 12/527,922

(22) PCT Filed: Mar. 7, 2008
(86) PCT No.: PCT/IT2008/000154

§ 371 Date: Aug. 20, 2009

Related U.S. Application Data
(60) Provisional application No. 60/906,251, filed on Mar. 12, 2007.

(51) Int. Cl. A61B 17/08

(52) U.S. Cl. 606/151

(57) ABSTRACT

An anchor to attach a tissue/suture-material (T) into a bone (200) comprises a structure having a proximal summit (20) from which depart two prongs (30, 40) having the distal ends (31, 41) free, in which said anchor (10, 110, 210, 310, 410) can assume at least two operative configurations: a first “open” configuration wherein the distal ends (31, 41) of the prongs (30, 40) are positioned spaced from one another; and a second “closed” configuration wherein the distal ends (31, 41) of the prongs (30, 40) are positioned close to one another.

### TECHNICAL FIELD

This invention pertains to an anchor, a related system comprising said anchor and a sleeve, and some methods to utilize said anchor and/or said system with the aim to attach human tissue (i.e. a ligament and/or a tendon and/or other similar element) or to attach a suture-material (i.e. a suture-material previously sutured to a human tissue) into a bone, particularly suitable for orthopedic surgery to attach tendons and ligaments to the humeral head.

### BACKGROUND OF THE INVENTION

At present, different typologies of anchors with related systems and related methods are known to attach human tissue or suture-material with respect to a bone.

These known techniques provide a series of disadvantages, mainly they are expensive and laborious to realize and require very long operative time.

### DISCLOSURE OF THE INVENTION

The object of the present invention is therefore to overcome the aforementioned drawbacks.

The invention, which is characterized by the claims, resolves the problem of providing an anchor for attaching a tissue or a suture-material into a bone, in which said anchor is characterized in that it comprises a structure having a proximal summit from which depart two prongs having the distal ends free and in that said anchor features at least two operative configurations: —a first “open” configuration, wherein the distal ends of the prongs are positioned distant from one another; and—a second “closed” configuration, wherein the distal ends of the prongs are positioned close to one another.

The invention, which is characterized by the claims, resolves the problem of creating a system to attach a tissue or a suture-material into a bone, in which said system is characterized in that it is comprised of said anchor and a sleeve.

The invention, which is characterized by the claims, resolves the problem of providing a method to attach a tissue or a suture-material into a bone, in which said method is characterised by the fact that it utilizes said anchor or said system, and by the fact that said method comprises the following operations: a)—the anchor is positioned in “open” configuration with the two distal ends distant from one another; b)—a portion of tissue or of suture-material is placed between the two distal ends distant from one another; c)—the distal ends of said anchor are closed with the aim to perforate with a tip the tissue or the suture-material positioning the same tissue or suture-material hooked within said anchor; d)—the same anchor and the related hooked tissue or suture-material is dragged towards a point wherein the tissue or suture-material shall be attached; e)—the anchor in “closed” configuration is attached to the bone in the fixation point.

### BRIEF DESCRIPTION OF THE DRAWINGS

Further features and advantages of this invention will result more evident in the following description of some of its preferred embodiments, here given merely as illustrative and not limitative example, made with reference to the figures of the enclosed drawings of which:

FIG. 1 and FIG. 1A illustrate the anchor subject of this invention in “open” configuration;

FIG. 2 illustrates the anchor of FIGS. 1 and 1A in “closed” configuration;

FIG. 2A illustrates the anchor of FIG. 2 in relation to the section line 2A-2A;

FIG. 2B illustrates the detail 2B of FIG. 2;

FIGS. 3, 3A, 3B, 3C, 3D and 3E illustrate some operative phases to attach tissue or suture-material using the anchor of FIGS. 1 and 2;

FIGS. 4, 4A, 4B and 4C illustrate a system (anchor+sleeve) subject of this invention and a related method to attach tissue or suture-material into a bone;

FIG. 5 illustrates the anchor subject of this invention in a second alternative embodiment;

FIG. 6 illustrates the anchor subject of this invention in a third alternative embodiment;

FIG. 6A illustrates an embodiment with a reinforced core;

FIG. 7 illustrates the anchor subject of this invention in a fourth alternative embodiment;

FIG. 8 illustrates a second embodiment for the sleeve;

FIG. 9 illustrates a third embodiment for the sleeve;

FIG. 10 illustrates a fourth embodiment for the sleeve.

FIG. 11 illustrates the anchor subject of this invention in a fifth alternative embodiment and in “open” configuration.

FIG. 11A illustrates the anchor of FIG. 11 in “closed” configuration;

FIG. 12 illustrates the anchor of FIG. 11.

### DESCRIPTION OF THE PREFERRED EMBODIMENT

### Description of the Anchor

### 1° Embodiment

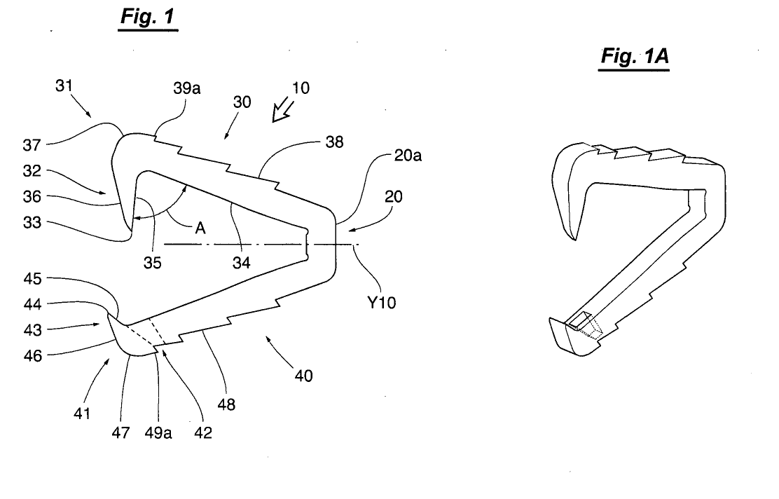

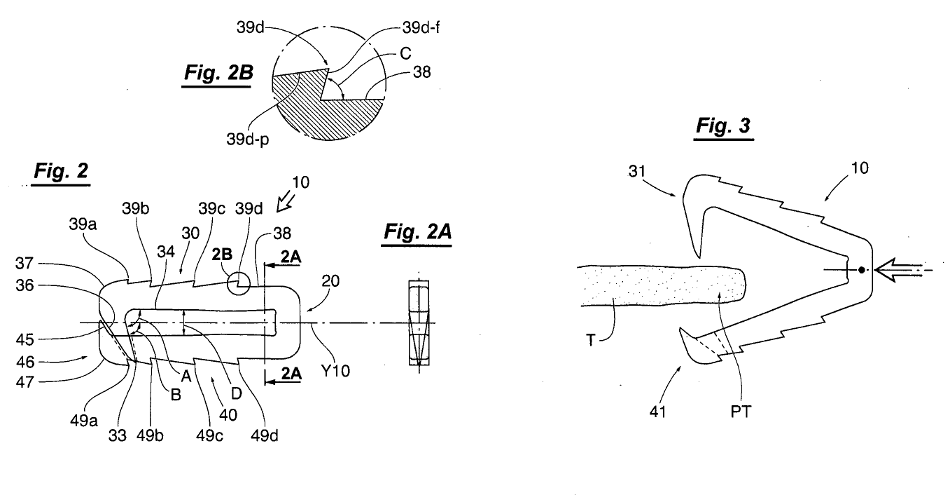

With reference to FIGS. 1 and 2, the anchor 10 subject of this invention, in relation to a longitudinal axis Y10, presents itself as a threadlike structure with cross section preferably square (see FIG. 2A) and moreover presents a configuration at “U” and/or “V” shape.

In the illustrated exemplifying form, such anchor 10 comprises a summit or vertex 20, a first prong 30 and a second prong 40, wherein said summit 20 is preferably made up of a portion 20a extending crosswise with respect to such longitudinal axis Y10, wherein the two prongs 30 and 40 are carried to the opposite ends of said portion 20a.

Said anchor 10 can be manufactured with different materials: metal, plastics, compounded, organic, etc., without going out of the inventive concepts of this invention.

In the present exemplifying embodiment, said anchor 10 is made with a plastic material, elastic, flexible, with a good resistance grade to traction and compression, and a reasonable hardness, which will be afterwards dissolved-absorbed by the bone.

Said anchor 10, substantially but not restrictively, can assume at least two operative configurations, a first “open” configuration (see FIG. 1) wherein the distal end portions 31 and 41 of the prongs 30 and 40 are positioned distant from one another, and a second “closed” configuration (see FIG. 2) wherein the distal end portions 31 and 41 of said “U” are positioned to meet one another.

With reference to FIG. 2, in the “closed” configuration said anchor 10 presents substantially a “0” shape wherein, the proximal or summit end is defined from the base 20 and the lower distal end of said “0” is obtained by means of the two distal ends 31, 41 of the related prongs 30, 40 positioned close to one another, with said prongs 30 and 40 spaced from one another with a pre-determined distance D, with the purpose to form a slot D between said two prongs 30 and 40 in said second “closed” configuration.

Preferably, near the distal end 31 of the first prong 30 is featured a tip 32, which has a sharp point 33, which features a conformation particularly suitable to perforate the tissue/suture-material, wherein said point 33 is turned towards the distal end 41 of the second prong 40, wherein said tip 32 is aimed to define a first internal profile 35 and a first external profile 36.

In the preferred embodiment, the tip 32 is hook shaped and said first internal profile 35, with respect to the internal profile 34 of the first prong 30, configures an angle “A” preferably less than 90° (i.e. approx)83° and, preferably, see FIG. 2, when said anchor 10 is in “closed” configuration, the same first internal profile 35, with respect to the longitudinal median axis Y10, configures an angle B less than 90°, (i.e. approx 83°.

The external profile 38 of the first prong 30, near its distal end 31, features a rounding-off/bevel 37 and, always preferably, near said rounding-off 37 and/or in the area of greater width, are included the first means of external restraining 39a, 39b, etc., aimed to allow an insertion of said anchor 10 in “closed” configuration in a hole, with unidirectional and anti-return insertion.

With reference to the second prong 40, near the portion of distal end 41 is positioned a slot 42, preferably of pass-through type, shaped to receive the distal portion of the tip 32, with the purpose of obtaining a coupling between the tip 32 and the slot 42.

Preferably, said slot 42 defines an inlet countersunk mouth whose function is addressing-guiding the insertion of tip 32 inside said slot 42, which must be of adequate size to allow the point 33 to protrude from said slot 42 (see FIG. 2).

Optionally, near the distal end of the second prong 40 could be included a hoe shaped tapered block 43, having the related sharp edge 44 pointing towards the distal end 31 of the first prong 30, defining a second internal profile 45 and a second external profile 46, wherein the second internal profile 45 carries out the function of addressing-guiding the sharp point 33 of the tip 32 inside the slot 42.

Preferably, said internal profile 45 is positioned (see FIG. 2) against the first external profile 36 of tip 32 when said anchor 10 is in “closed” configuration, with the aim to carry out a thrust-block function.

Preferably, the external profile 48 of the second prong 40, near its distal end 41, presents a rounding-off/bevel 47 and, near said rounding-off/bevel 47 and/or in an area of greater width, are included second means of external restraining 49, as for example a tooth 49a, for the reasons that will be more readily apparent hereinafter.

Said anchor 10, along the external profile/perimeter 38 and 48 of the two prongs 30 and 40 can include preferably other first and/or second external restraining means, 39 and/or 49, in this specific case some restraining teeth 39a-39b-39c-39d and 49a-49b-49c-49d.

Preferably, said restraining teeth 39a-39b-39c-39d and 49a-49b-49c-49d (see FIG. 2B) feature an inclined plane 39d-p and a striker face 39d-f, wherein said striker face 39d-f is positioned at an angle with respect to the external profile 38 of prong 30 with angle C less than 90°.

Method to Attach Anchor and Tissue/Suture-Material within a Hole in a Bone

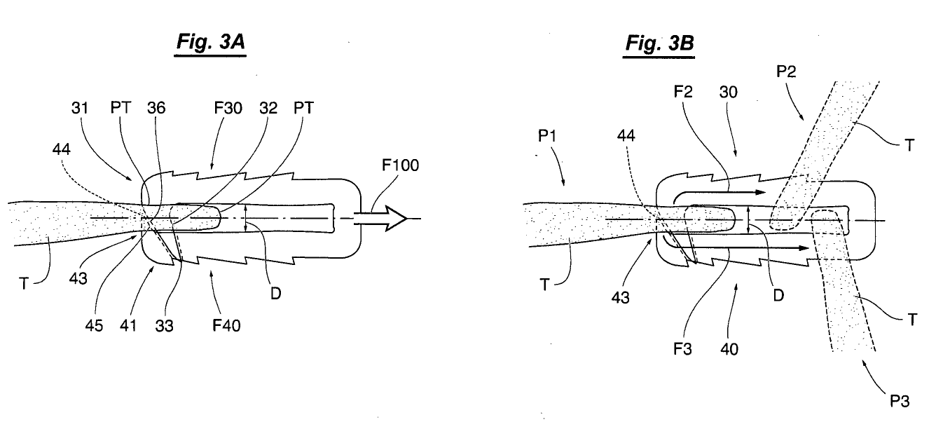

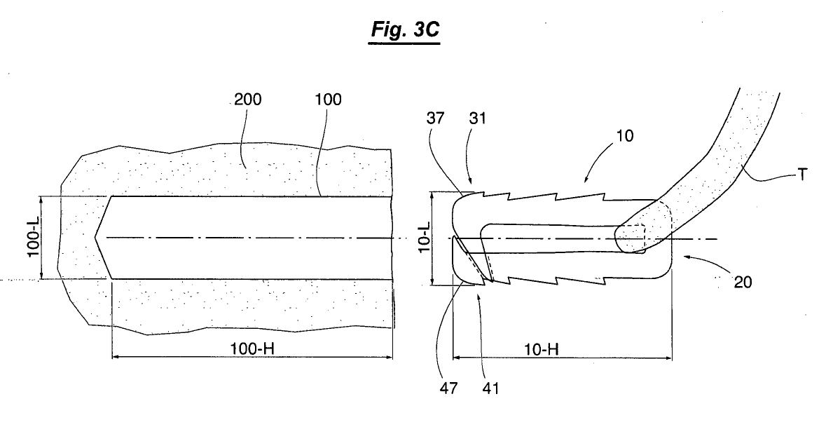

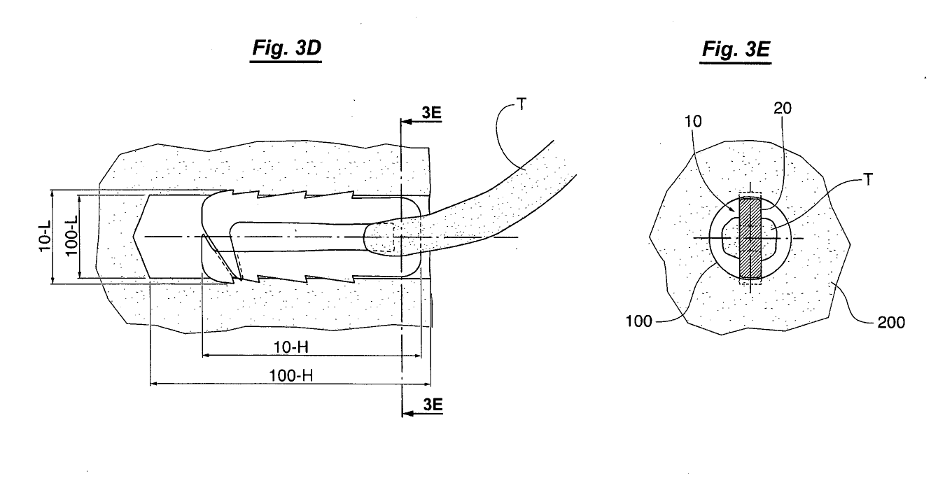

With reference to FIGS. 3-3A-3B-3C they illustrate the steps related to a first method to attach a tissue/suture-material T into a bone 200, utilizing the anchor 10 subject of this invention.

With reference to FIG. 3, with the anchor 10 in open position a portion PT of the tissue/suture-material T is arranged within said anchor 10 and between the two distal ends, 31 and 41.

The anchor 10 closing is carried out (see arrows F30 and F40 in FIG. 3A) and, during such closing operation, the point 33 of the tip 32 perforates the tissue/suture-material T and, following a further closing of the distal ends 31 and 41, the tip 32 inserts within the slot 42, with the aid of second internal profile 45 of the hoe 43 that acts as deviator, wherein the edge 44 of said hoe 43 operates as counter-perforator to facilitate the perforation operation. Further to the perforation made (anchor in “closed” configuration) said anchor 20 has the second internal profile 45 of said hoe 43 placed laterally and/or near the external profile 36 of the tip 32.

In such context, it is specified that the edge 44 of the hoe 43 assumes different shapes and counter-perforation angles in relation to the tissue/suture-material to be perforated.

In such manner, the tissue/suture-material T results positioned (hooked and/or tight and/or closed and/or locked) within said anchor 10 and, therefore, with said anchor 10 in a “closed” configuration, it is possible to drag the same anchor 10 and the related tissue/suture-material T towards a point wherein the tissue/suture-material T shall be attached.

In such context (see FIG. 3A) please notice that during the traction-translation operations, applied for example by means of a force F100, the portion of tissue/suture-material PT remains in grasp within said anchor 10 due to the tip 32.

Note that it is possible to apply a great traction force, due to the special hook shaped configuration of tip 32 and the coupling of tip 32 with the slot 42 and the presence of block-hoe 43, which internal profile 45, during the traction, can act as a thrust-block, if the external profile 36 of the tip 32 enters into contact with said internal profile 45 of hoe 43.

Furthermore (see FIG. 3B) during the subsequent operations, as more readily apparent hereinafter, the tissue/suture-material T can slide from the position P1 to the position P2, sliding along the first prong 30, arrow F2, or from the position P1 to the position P3, sliding along the second prong 40, arrow F3 and, in this latter case, the “hoe shape” of tapered block 43 with sharp edge 44 facilitates said operation (as also the operations of the previous perforation), since said edge 44 wedges-in in the perforation previously made by tip 32.

The tissue/suture-material T can easily move from the position P1 to the position P2 or P3 thanks to the pre-determined distance D between said two prongs 30 and 40 when the anchor is in its “closed” configuration.

To attach the anchor 10 with tissue/suture-material T into the bone (see FIG. 3C) a hole 100 is made in the bone 200 at the location wherein it is desired to attach the tissue/suture-material, and said hole 100 presents a deepness 100-H and diameter 100-L related to the height 10-H and to the width 10-L of the anchor 10 in closed configuration.

Therefore (see FIG. 3D) the distal ends 31-41 of the anchor 10 in “closed” configuration with related attached tissue/suture-material T are inserted in the mentioned hole 100. Rounding-offs/bevels 37 and 47 facilitate and self-center said insertion.

Furthermore, if necessary, during said insertion the attached tissue/suture-material T is pushed by the upper edge of hole 100 towards summit 20 of anchor 10 sliding along the prongs 30 or 40.

Preferably, but not restrictively, the hole 100 presents a diameter 100-L slightly less than the width 10-L of the anchor 10 and preferably but not restrictively, a depth 100-H longer than the height 10-H of the anchor 10.

The anchor 10 and the related tissue/suture-material T are inserted in said hole 100 (see FIG. 3D) achieving a coupling anchor-hole, 10-100, with graduated and wanted interference in relation to the load that the tissue/suture-material will support.

Optionally, said anchor 10 can be pushed within the same hole 100 with variable insertion depth to obtain a desired tension of the tissue/suture-material T, to obtain a desired length of tissue/suture-material placed out of the hole 100 and to obtain a desired length of tissue/suture-material placed into the hole 100.

Preferably, the anchor 10 is completely inserted within the hole 100 to avoid the anchor protruding from the hole summit.

Further to the above-mentioned operations, (see FIGS. 3D and 3E) the tissue/suture-material T results positioned in grasp within said anchor 10, positioned near the summit or vertex 20 and, the same anchor 10, is positioned within the mentioned hole 100 with the teeth 39 and 49 pressing against the walls of the same hole 100, making stable said coupling and achieving, as desired, in a quick and simple way, the fastening of the tissue/suture-material T into a bone 200.

Please note that the tissue/suture-material T is positioned within the bone at a desired depth and it is possible to predetermine the length of tissue/suture-material T that will be inserted within the bone in view of the future osteogenesis.

System (Anchor and Sleeve) and Method to Attach Tissue/Suture-Material into a Bone

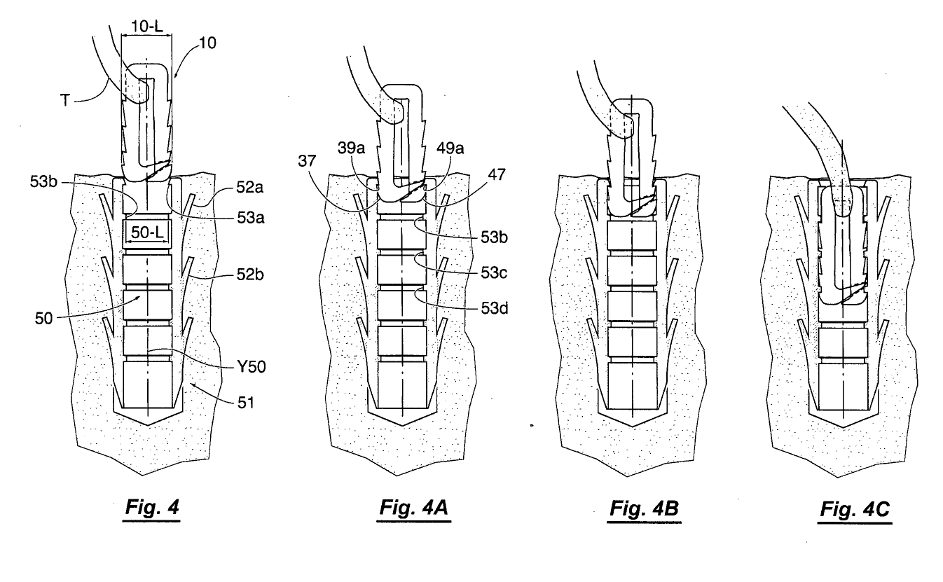

With reference to FIG. 4-4A-4B-4C the anchor 10 described above can also be utilized in combination with a sleeve 50 as illustrated in FIG. 4, defining a system 10-50 to attach tissue/suture-material into a bone.

With reference to FIG. 4, sleeve 50 extends along a longitudinal axis Y50 and, substantially, it is a hollow cylinder, with distal end 51 preferably tapered, with external shell equipped of third restraining means 52, as for example the flexible tongues 52a, 52b, etc.

In the internal part of the hollow sleeve 50 are included at least one or more third restraining means 53, with the aim to cooperate as a ratchet with the first and/or second external restraining means, 39-49 of anchor 10, as for example some circumferential teeth, 53a, 53b, 53c, 53d, etc., positioned axially distanced, with a minimum calibrated diameter 50-L slightly less than the width 10-L of the teeth 39-49 of the anchor 10, with the aim to be able to insert the anchor 10 in “closed” configuration within said sleeve 50 and achieve a ratchet between the external teeth 39-49 and the internal circumferential tooth 53 of sleeve 50, with unidirectional and anti-return insertion, preferably step-by-step, as better described hereinafter.

With such system 10-50, sleeve 50 is inserted in a hole made in the bone and distal ends 31-41 of anchor 10 in “closed” configuration with attached tissue/suture-material T are inserted in the mentioned sleeve 50 with rounding-offs/bevels 37-47 facilitating and self-centering said insertion.

During said insertion, tissue/suture material T, lodged within the anchor 10, is pushed by the upper edge of sleeve 50, towards the summit of anchor 10, sliding along the prongs 30 or 40 and, therefore, towards the summit 20.

Preferably, anchor 10 includes two first teeth 39a and 49a, positioned near the distal end of the same anchor 10, for example near the two rounding-offs 37 and 47, and the sleeve 50 includes a first circumferential tooth, 53a, see FIG. 4, positioned near the proximal or upper end of the same sleeve 50, in such a way to achieve an anti-return restraining for the anchor 10 even if only a small distal portion of the same anchor 10 is inserted within the sleeve 50, as illustrated on FIG. 4A.

Once anchor 10 is partially positioned within the sleeve and inhibited to withdraw, it is suggested to completely insert anchor 10 within sleeve 50, as illustrated on FIG. 4C, achieving a stable and secure attachment of tissue/suture-material T into the bone.

As for the previous method, please note that the tissue/suture material T pierced-hooked by the anchor 10 is deeply inserted into the bone, and more particularly at a desired depth, chosen/decided by an operator, during the surgical operation. In this manner, it is possible both to determine the length (quantity) of tissue to be inserted into the bone, in view of the future osteogenesis and/or to determine the tension of the same tissue/suture material T.

Note that sleeve 50 can be made of different materials, metallic, plastic, compound, organic, etc., without going out of the inventive concepts of this invention.

In the present embodiment, said sleeve 50 is made of a plastic material, preferably elastic, flexible, with optimal resistance to traction and compression, and reasonable hardness, which will be afterwards dissolved/absorbed by the bone.

### 2^{nd} Embodiment of the Anchor

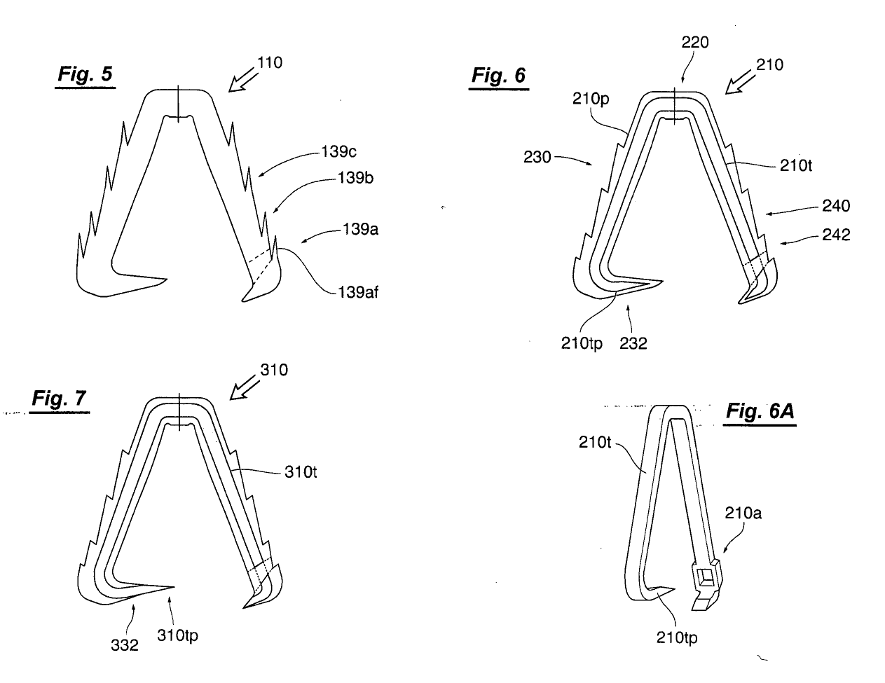

With reference to FIG. 5, it is illustrated a second anchor embodiment, here indicated with reference 110, defining external restraining means 139a, 139b, 139c, etc., of tongue type, see 139-af, wherein each tongue is capable of flexing internally.

### 3^{rd} Embodiment of the Anchor

With reference to FIG. 6, it is illustrated a third anchor embodiment, here indicated with the reference 210, including a threadlike core 210t, illustrated in an exemplifying view on FIG. 6A, which is preferably made of an elastic metallic material, as for example a filament of nitinol.

Said core 210t extends preferably along the summit 220 and along the prongs 230 and 240 of said anchor 210, with the aim to reinforce and maintain the structural integrity of said anchor 210 in the event of mechanical failure of the plastic layer.

Optionally, said core 210t can be extended also towards and within tip 232, portion 210tp, enhancing the mechanical properties of said tip 232, and can also be extended towards and around the slot 242 defining a reinforced slot 210a.

### 4^{th} Embodiment of the Anchor

With reference to FIG. 7, it is illustrated a fourth embodiment, here indicated with the reference 310, wherein a core 310t extends within and out of tip 332, presenting a portion-point 310tp projecting externally and defining a metallic perforation tip 332.

With such metal insert it is possible to achieve the characteristics above explained in relation to FIG. 6 together with a tip 332 having a metal point 310tp that presents optimal perforation capabilities.

### 5^{th} Embodiment of the Anchor

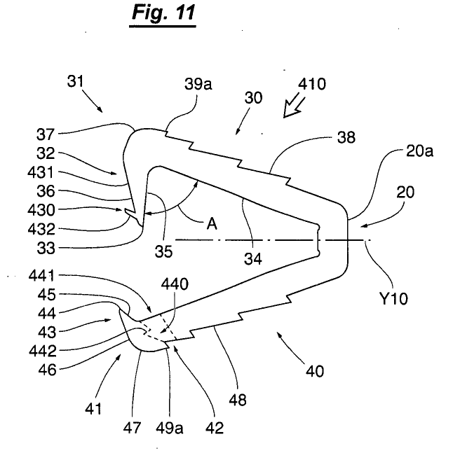

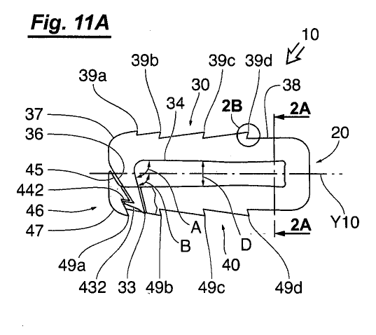

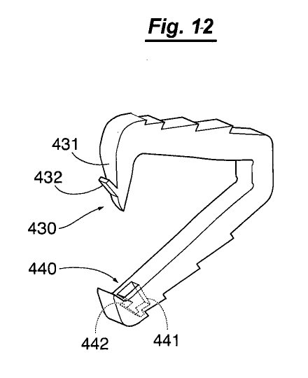

With reference to FIGS. 11, 11A, and 12, they illustrate a fifth embodiment of the anchor, here indicated with the reference 410, further comprising fastening means 430-440 to fasten the two distal ends 31 and 41 of the prongs 30 and 40 when anchor 10 is in “closed” configuration.

Said fastening means 430-440 are positioned on the distal ends, 31 41, of the two prongs, 30 and 40, and they comprise an automatic coupling system.

More in particular, in the illustrated exemplifying form, said fastening means 430-440 comprise a harpoon 431, with flexible tooth 432, at the distal end of the first prong 30, and a receptacle 441, with ratchet tooth 442, at the distal end of the second prong 40, so that when the distal ends 31 and 41 are closed, see FIG. 11A, with the harpoon 431 inserted into the receptacle 441, an automatic fastener will be obtained by engagement of the flexible tooth 432 with the ratchet tooth 442.

With reference to the embodiment above described, anchor 410, the operation in which the distal ends 31 41 of anchor 10 are closed, includes a further operation wherein the distal ends 31 and 41 of the prong 30 and 40 of the anchor 410 are fastened together.

Preferably, to form said fastening means 430-440, the tip 32 of the first anchor is equipped with a flexible tooth 432 and the slot 42 of the first anchor is equipped with ratchet tooth 442.

### 2^{nd} Embodiment of the Sleeve

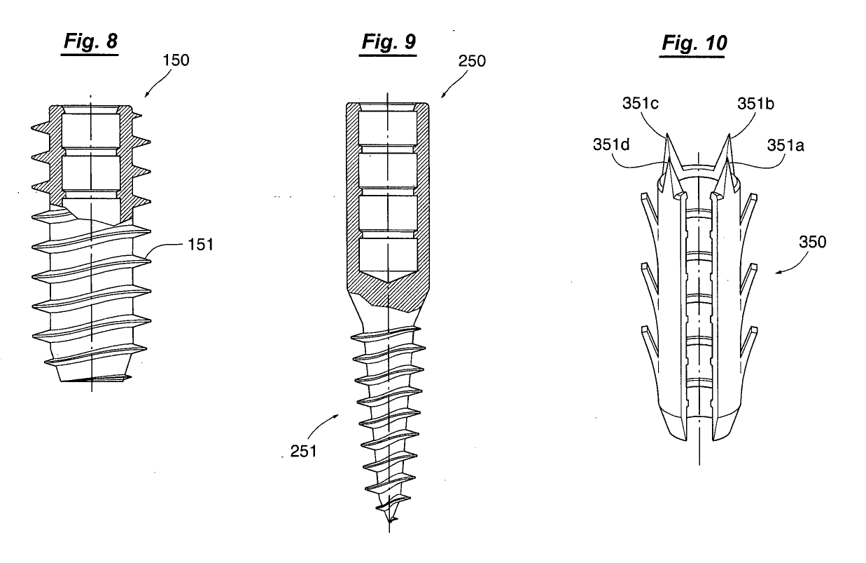

With reference to FIG. 8, it illustrates a second embodiment of the sleeve, here indicated with 150, defining on its external shell a threading 151.

### 3^{rd} Embodiment of the Sleeve

With reference to FIG. 9, it illustrates a third embodiment of the sleeve, here indicated with 250, defining near its distal end a screw shaped appendix 251.

### 4^{th} Embodiment of the Sleeve

With reference to FIG. 10, it illustrates a fourth embodiment of the sleeve, here indicated with 350 defining on its proximal end one or more engaging means, as for example some tips (cuspids) 351a, 351b, etc., which are aimed at restraining tissue/suture-material T, after the insertion of the anchor with lodged tissue within said sleeve 350.

The description of the anchors, of the related systems comprising anchors and sleeves and the related methods to attach a tissue/suture material T into a bone, are given merely as exemplificative and not limitative and, therefore, it seems evident that modifications or variations can be applied to them, suggested by experience and/or utilisation and/or use.

The following claims make up also an integrative part of the above expressed description.

1-93. (canceled)

94. An anchor for attaching tissue or suture-material into a bone, in which the anchor comprises a structure having a proximal summit from which depart two prongs having the distal ends free, in which the anchor can assume at least two operative configurations,
a first open configuration, wherein the distal ends of the prongs are positioned spaced from one another;
a second closed configuration, wherein the distal ends of the prongs are positioned close to one another wherein near the distal end of a first prong protrudes a tip serving for perforating the tissue/suture material with a point turned toward the distal end of the second prong, and wherein the second prong, near its portion of distal end defines a slot serving for receiving the distal portion of the tip carried by the first prong.

95. The anchor defined in claim 94 wherein the open configuration with the distal ends of the prongs positioned spaced is assumed normally if external forces are not applied.

96. The anchor defined in claim 94 wherein the closed configuration with the distal ends of the prongs positioned close to one another is assumed by the anchor if external forces are applied.

97. The anchor defined in claim 94 wherein in the closed configuration the anchor conforms to a 0 shape wherein the distal end of the 0 is obtained by means of the two distal ends of the related prongs positioned close to one another.

98. The anchor defined in claim 94 wherein in the closed configuration the anchor conforms to a 0 shape wherein the distal end of the 0 is obtained by means of the two distal ends of the related prongs intersecting one another.

99. The anchor defined in claim 94 wherein in the closed configuration the anchor conforms to a 0 shape wherein the distal end of the 0 is obtained by means of the two distal ends of the related prongs coupling to one another.

100. The anchor defined in claim 94 wherein in the closed configuration the two prongs are spaced from one another at a pre-determined distance.

101. The anchor defined in claim 94, further comprising:
fastening means for fastening in closed configuration the two distal ends of the prongs.

102. The anchor defined in claim 101 wherein the fastening means are positioned at the distal ends of the two prongs.

103. The anchor defined in claim 101 wherein the fastening means comprise an automatic coupling/fastening system.

104. The anchor defined in claim 101 wherein the fastening means comprise a harpoon with at least a flexible tooth at the distal end of the first prong and a receptacle with a ratchet tooth at the distal end of the second prong.

105. The anchor defined in claim 94 wherein the tip defines a first internal profile and the internal profile with respect to the internal profile of the related prong configures an angle less than 90°.

106. The anchor defined in claim 94 wherein the tip defines a first internal profile and that when the anchor is in closed configuration the same first internal profile configures an angle less than 90° with respect to the longitudinal median axis.

107. The anchor defined in claim 94 wherein the first prong, near its distal end, along its external profile, defines a rounding-off or bevel.

108. The anchor defined in claim 94 wherein the first prong, along its external profile, near its distal end, defines first external restraining means.

109. The anchor defined in claim 108 wherein the first restraining means are positioned near the rounding-off or bevel.

110. The anchor defined in claim 108 wherein the first external restraining means serve for allowing an insertion of the anchor in closed configuration in a hole with unidirectional and anti-return advancing.

111. The anchor defined in claim 94 wherein the slot defines an inlet countersunk mouth serving to address-guide tip insertion within the slot.

112. The anchor defined in claim 94 wherein the second prong, near its portion of distal end, defines a tapered block with the related edge turned toward distal end of first prong.

113. The anchor defined in claim 94 wherein the second prong, near its distal end defines a second internal profile serving for address-guiding the point of the tip carried by first prong within a slot on second prong.

114. The anchor defined in claim 94 wherein second prong, near its distal end defines a second internal profile which is orientated and conformed in such a way to position laterally, near or against, the first external profile of the tip carried to the distal end of the first prong when the anchor is in closed configuration.

115. The anchor defined in claim 94 wherein the second prong near its distal end defines along its external profile a rounding-off or bevel.

116. The anchor defined in claim 94 wherein on the second prong along its external profile and near its distal end are positioned second external restraining means.

117. The anchor defined in claim 116 wherein the second restraining means are positioned near the rounding-off or bevel.

118. The anchor defined in claim 116 wherein along the external profile of the first prong or along the external profile of the second prong are positioned one or more first or second external restraining means.

119. The anchor defined in claim 118 wherein the external restraining means allow an insertion of the anchor in closed configuration in a hole with unidirectional and anti-return advancing.

120. The anchor defined in claim 116 wherein the first or second external restraining means comprise a tooth protruding with respect to the external profile of the prongs.

121. The anchor defined in claim 116 wherein the first or second external restraining means comprise a tooth that presents a striker face positioned at an angle with respect to the external profile of the related prong.

122. The anchor defined in claim 116 wherein the first or second external restraining means comprise a restraining tooth comprising a striker face and an inclined plane serving for facilitating the insertion of the anchor within a hole.

123. The anchor defined in claim 122 wherein the striker face with respect to the external profile of the related prong configures an angle less than 90°.

124. The anchor according claim 116 wherein the first or second restraining means comprise flexible tongues.

125. The anchor defined in claim 94 wherein the two prongs are elastically flexible.

126. The anchor defined in claim 94, further comprising
an internal core.

127. The anchor defined in claim 126 wherein the internal core is made of metallic material.

128. The anchor defined in claim 126 wherein the internal core extends along the summit and along the prongs of the anchor.

129. The anchor defined in claim 126 wherein the internal core extends toward and within the tip.

130. The anchor defined in claim 126 wherein the internal core extends toward and around the slot and defines a reinforced slot.

131. The anchor defined in claim 126 wherein the internal core extends within and past the tip so as to define a portion-point projecting externally and serving for defining the perforation point for the same tip.

132. The anchor defined in claim 94 wherein the tip is equipped with a flexible tooth to form fastening means for fastening in closed configuration the two distal ends of prongs.

133. The anchor defined in claim 94 wherein the slot is equipped with a ratchet tooth to form fastening means for fastening in closed configuration the two distal ends of prongs.

134. A system for attaching tissue or suture-material into a bone, further comprising
an anchor defined in claim 94 and a sleeve.

135. The system defined in claim 134 wherein the sleeve extends along a longitudinal axis and presents itself as a hollow cylinder serving for receiving in its interior the mentioned anchor in closed configuration.

136. The system defined in claim 134 wherein the sleeve is tapered at its distal end.

137. The system defined in claim 134 wherein the sleeve is equipped on its outside surface with third external restraining means.

138. The system defined in claim 137 wherein the third external restraining means comprise flexible tongues.

139. The system defined in claim 137 wherein the third external restraining means comprise a screwthread.

140. The system defined in claim 134 wherein the sleeve includes a screwthread on its outside surface.

141. The system defined in claim 134 wherein the sleeve includes near its distal end an appendix serving for configuring a screw.

142. The system defined in claim 134 wherein the sleeve includes near its proximal end one or more restraining teeth.

143. The system defined in claim 134 wherein the sleeve comprises one or more internal restraining means.

144. The system defined in claim 143 wherein the internal restraining means comprise one or more internal circumferential teeth.

145. The system defined in claim 143 wherein the internal restraining means serve for allowing the insertion of the anchor in closed configuration within the sleeve and serve for restraining the anchor within the sleeve after its insertion.

146. The system defined in claim 144 wherein the one or more internal circumferential teeth have a calibrated minimum diameter slightly smaller than the width of the anchor in closed configuration.

147. The system defined in claim 144 wherein the one or more internal circumferential teeth have a calibrated minimum diameter slightly inferior respect to the width of the anchor so as to be capable to insert the anchor in closed configuration within the sleeve and so as to obtain subsequently to the anchor insertion a ratchet between the external restraining means of the anchor and the third internal circumferential restraining means of the sleeve.

148. The system defined in claim 134 wherein the sleeve comprises a first internal circumferential restraining mean near its upper end, serving for engaging the restraining means positioned near the distal end of the anchor in closed configuration.

149. A method for attaching tissue or suture-material into a bone wherein is utilized an anchor comprising at least a proximal summit, a first prong and a second prong, wherein the anchor carries at the distal end of a first prong a perforating tip, and it comprises the following steps:
a) the anchor is positioned in an open configuration with the two distal ends spaced from one another;
b) a portion of tissue/suture-material is placed between the two distal ends.
c) the distal ends of the anchor are closed so as to perforate with the tip the tissue/suture-material positioning the same tissue/suture-material lodged within a space of the closed anchor;
d) the anchor and the related hooked tissue/suture-material is dragged toward a point wherein the tissue/suture-material shall attach; and
e) the anchor in closed configuration is attached to the bone at the fixation point.

150. The method defined in claim 149 wherein the anchor can assume in step c)
a second closed configuration, wherein the distal ends of the prongs are positioned close to one another wherein near the distal end of a first prong protrudes a tip serving for perforating the tissue/suture material with a point turned toward the distal end of the second prong, and wherein the second prong, near its portion of distal end defines a slot serving for receiving the distal portion of the tip carried by the first prong.

151. The method defined in claim 149 wherein operation c) includes a further operation cl) wherein the distal ends of the prongs of the anchor are fastened together.

152. The method defined in claim 149 wherein operation e) includes a special operation e1) wherein the distal ends of the anchor are inserted, in closed configuration and close to one another, within a hole made in the bone.

153. The method defined in claim 149 wherein the operation e) includes a special operation e2) wherein the distal ends of the anchor are inserted, in closed configuration and close to one another, within a sleeve which is positioned within a hole made in the bone.

154. The method defined in claim 149 wherein the tissue/suture-material hooked at the distal ends is moved toward the proximal summit of the anchor by sliding the tissue/suture-material along a first prong.

155. The method defined in claim 149 wherein the tissue/suture-material hooked at the distal ends is moved toward the proximal summit of the anchor by sliding the tissue/suture-material along a second prong.

156. The method defined in claim 149 wherein the hole made in the bone has a diameter slightly smaller than the width of the anchor in closed configuration.

157. The method defined in claim 149 wherein the hole made in the bone presents a depth longer than the height of the anchor.

158. The method defined in claim 149 wherein the anchor is pushed within the hole with insertion depth variable to obtain a desired or correct tension of the tissue/suture-material.

159. The method defined in claim 149 wherein the anchor is pushed within the hole with insertion depth variable to obtain a desired or correct length of tissue/suture-material placed out of the hole.

160. The method defined in claim 149, characterized by the fact the anchor is completely inserted within the hole so as to avoid the anchor overhanging with respect to the hole summit.

161. The method defined in claim 149 wherein the anchor is pushed within the hole with variable insertion depth so as to determine the quantity of tissue that it is desired to incorporate within the bone.

162. The method defined in claim 149 wherein a sleeve is used with the anchor.

163. The method defined in claim 162 wherein the anchor is pushed within the sleeve with insertion depth variable to obtain a desired/correct tension of the tissue/suture-material.

164. The method defined in claim 162 wherein the anchor is pushed within the sleeve with insertion depth variable to obtain a desired/correct length of tissue/suture-material placed out of the sleeve.

165. The method defined in claim 162 wherein the anchor is completely inserted within the sleeve so as to avoid the anchor overhanging with respect to the sleeve summit.

166. The method defined in claim 162 wherein the anchor is pushed within the sleeve with variable insertion depth so as to determine the quantity of tissue that it is desired to incorporate within the sleeve.
